(19) United States
(12) Patent Application Publication
(10) Pub. No.: US 2021/0345859 A1
(43) Pub. Date: Nov. 11, 2021

(54) INSERTION DEVICE AND ENDOSCOPE

(71) Applicant: OLYMPUS CORPORATION, Tokyo (JP)

(72) Inventor: Tatsuhiko OKITA, Tokyo (JP)

(73) Assignee: OLYMPUS CORPORATION, Tokyo (JP)

(21) Appl. No.: 17/380,138

(22) Filed: Jul. 20, 2021

Related U.S. Application Data
(63) Continuation of application No. PCT/JP2019/002501, filed on Jan. 25, 2019.

(51) Int. Cl. A61B 1/005; A61B 1/00; A61B 1/018

(52) U.S. Cl. CPC A61B 1/0058; A61B 1/0008; A61B 1/018; A61B 1/00039

(57) ABSTRACT

An insertion device includes: an insertion portion; a shape memory alloy tube provided in the insertion portion such that a distal end is located on a side of one end of the insertion portion and a proximal end is located on a side of another end of the insertion portion, the shape memory alloy tube being configured to serve as a conduit through which a refrigerant flows from a side of the distal end toward a side of the proximal end; a heater configured to heat the shape memory alloy tube; and a refrigerant supply tube configured to supply the refrigerant to the distal end of the shape memory alloy tube by causing the refrigerant to flow from the proximal end side toward the distal end side of the shape memory alloy tube, the refrigerant supply tube being disposed inside the shape memory alloy tube.

### CROSS REFERENCE TO RELATED APPLICATION

This application is a continuation application of PCT/JP2019/002501 filed on Jan. 25, 2019, the entire contents of which are incorporated herein by this reference.

### BACKGROUND OF THE INVENTION

### 1. Field of the Invention

The present invention relates to an insertion device and an endoscope including a rigidity variable device using a shape memory alloy.

### 2. Description of the Related Art

Insertion devices including insertion portions with flexibility that are to be inserted into insertion targets such as organisms and structures have been used in a medical field and an industrial field, for example, in order to carry out observation and treatment inside the insertion targets. The insertion devices include endoscopes.

For example, International Publication No. 2017/094085 discloses a device that changes the resistance to bending deformation (rigidity) of an insertion portion of an insertion device. The device disclosed in International Publication No. 2017/094085 can increase the rigidity of the insertion portion through heating of a shape memory alloy disposed in the insertion portion.

If a portion of the insertion portion with the rigidity to be increased is caused to move in an axial direction, it may become easy to move the insertion portion in a curved conduit of an insertion target.

### SUMMARY OF THE INVENTION

An insertion device according to an aspect of the invention includes: an insertion portion including one end and another end and configured to be inserted into an insertion target from a side of the one end; a shape memory alloy tube including a distal end and a proximal end and provided in the insertion portion such that the distal end is located on the side of the one end of the insertion portion and the proximal end is located on a side of the other end of the insertion portion, the shape memory alloy tube being configured to serve as a conduit through which a refrigerant flows from a side of the distal end toward a side of the proximal end; a heater configured to heat the shape memory alloy tube; and a refrigerant supply tube configured to supply the refrigerant to the distal end of the shape memory alloy tube by causing the refrigerant to flow from the side of the proximal end of the shape memory alloy tube toward the side of the distal end of the shape memory alloy tube, the refrigerant supply tube being disposed inside the shape memory alloy tube.

An endoscope according to an aspect of the invention includes: an insertion portion including one end and another end and configured to be inserted into an insertion target from a side of the one end; a shape memory alloy tube including a distal end and a proximal end and provided in the insertion portion such that the distal end is located on the side of the one end of the insertion portion and the proximal end is located on a side of the other end of the insertion portion, the shape memory alloy tube being configured to serve as a conduit through which a refrigerant flows from a side of the distal end toward a side of the proximal end; a heater configured to heat the shape memory alloy tube; and a refrigerant supply tube configured to supply the refrigerant to the distal end of the shape memory alloy tube by causing the refrigerant to flow from the side of the proximal end of the shape memory alloy tube toward the side of the distal end of the shape memory alloy tube, the refrigerant supply tube being disposed inside the shape memory alloy tube.

### BRIEF DESCRIPTION OF THE DRAWINGS

FIG. 1 is a diagram illustrating a configuration of an insertion device according to a first embodiment;

FIG. 2 is a diagram illustrating a configuration of a rigidity variable device according to the first embodiment;

FIG. 3 is a diagram illustrating a section of the rigidity variable device according to the first embodiment;

FIG. 4 is a diagram illustrating a state in which a feeding device has been caused to operate in the insertion device according to the first embodiment;

FIG. 5 is a diagram for explaining operations of the insertion device according to the first embodiment; and

FIG. 6 is a diagram illustrating a configuration of a rigidity variable device according to a second embodiment.

### DETAILED DESCRIPTION OF THE PREFERRED EMBODIMENTS

Hereinafter, preferred embodiments of the invention will be described with reference to the drawings. Note that in each drawing used in the following description, each component is illustrated to have such a size that the component can be recognizable in the drawing, each component is thus illustrated with a different scale, and the invention is not limited only to numbers of components, shapes of the components, ratios of sizes of the components, and relative positional relationships of the respective components described in these drawings.

### First Embodiment

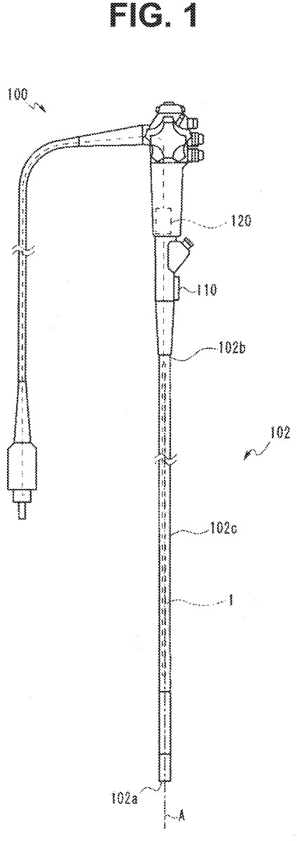

Hereinafter, an example of an embodiment of the invention will be described. An insertion device 100 illustrated in FIG. 1 includes an elongated insertion portion 102 that can be inserted into an insertion target such as a human body and has, in the insertion portion 102, a configuration for observing inside of the insertion target. In other words, the insertion device 100 is an endoscope in an example of the present embodiment. The insertion target into which the insertion portion 102 of the insertion device 100, which is an endoscope, is not limited to a human body, and may be another organism, or may be an artificial object such as a machine or a building. Further, the insertion device 100 is not limited to the form of the endoscope and may be any treatment instrument for carrying out medical treatment in a human body.

The insertion portion 102 has an elongated shape. In the following description, an axis in a longitudinal direction of the elongated insertion portion 102 will be referred to as a longitudinal axis A. The insertion portion 102 has a flexible tube portion 102c with flexibility. The flexible tube portion 102c may include a so-called bending portion that actively bending-deforms in accordance with an operation performed by a user of the insertion device 100. Note that although the linear longitudinal axis A is illustrated in FIG. 1, the longitudinal axis A deforms in accordance with bending deformation of the insertion portion 102.

In the following description, an end of the insertion portion 102 on a side on which the insertion portion 102 is inserted into the insertion target will be defined as one end 102a while an end that is opposite to the one end 102a will be defined as the other end 102b. In other words, the insertion portion 102 is inserted into the insertion target from the side of the one end 102a. Also, a direction following the longitudinal axis A will be referred to as an axial direction.

The insertion device 100 includes a rigidity variable device 1, an operation portion 110, a feeding device 120, and a control unit 130. The rigidity variable device 1 is disposed in at least a part of the flexible tube portion 102c of the insertion portion 102. The operation portion 110 and the control unit 130 are configurations for controlling operations of the rigidity variable device 1. The feeding device 120 is a configuration for feeding a refrigerant, which is described later.

The operation portion 110 includes a first switch 111 and a second switch 112. The first switch 111 and the second switch 112 are configurations for the user of the insertion device 100 to input instructions for controlling operations of the rigidity variable device 1. The first switch 111 and the second switch 112 are electrically connected to the control unit 130.

The control unit 130 controls operations of the rigidity variable device 1 and the feeding device 120, which will be described later, in accordance with inputs of instruction from the user to the operation portion 110. Note that electric power for causing the rigidity variable device 1 and the feeding device 120 to operate is supplied from an external device to which the insertion device 100 is connected. The external device is, for example, a video processor or a light source device. Note that the insertion device 100 may include a battery for supplying electric power to cause the rigidity variable device 1 and the feeding device 120 to operate.

Note that although the operation portion 110, the feeding device 120, and the control unit 130 are provided in the insertion device 100 in the example of the present embodiment, some or all of the operation portion 110, the feeding device 120, and the control unit 130 may be provided in an external device to which the insertion device 100 is connected.

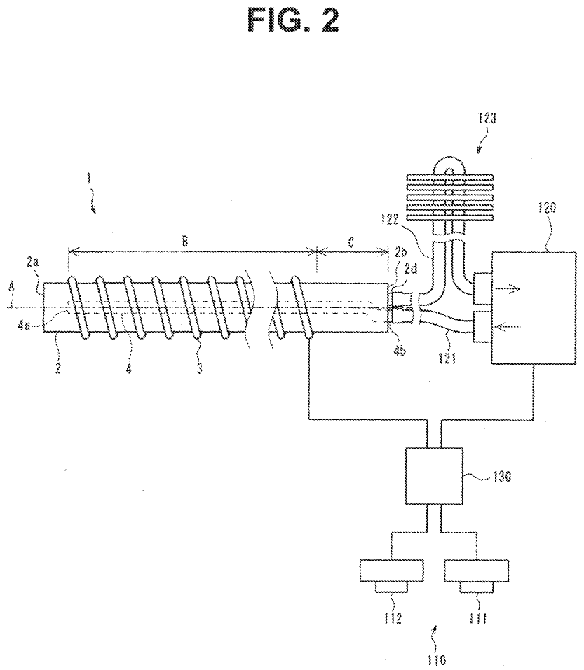

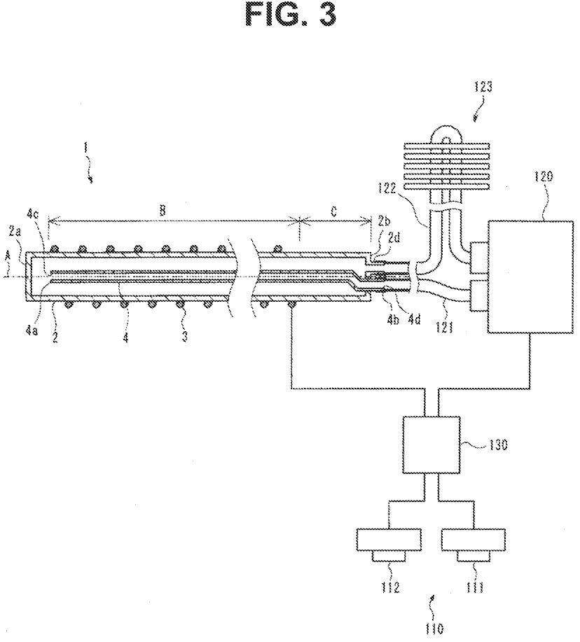

FIG. 2 is a diagram illustrating a configuration of the rigidity variable device 1. FIG. 3 is a sectional view of the rigidity variable device 1. The rigidity variable device 1 includes a shape memory alloy tube (hereinafter, referred to as an “SMA tube”) 2, a heater 3, and a refrigerant supply tube 4.

The SMA tube 2 is a pipe made of a shape memory alloy. The SMA tube 2 is disposed inside the flexible tube portion 102c of the insertion portion 102 along the longitudinal axis A. In other words, the SMA tube 2 has an elongated outer shape in the axial direction. The SMA tube 2 includes an inner space extending in the axial direction. The SMA tube 2 deforms with the bending deformation of the flexible tube portion 102c. Note that although the longitudinal axis A and the SMA tube 2 overlap each other in the drawing, the longitudinal axis A and the SMA tube 2 may be separated from each other.

Detailed description of the shape memory alloy will be omitted since the shape memory alloy is a known technique. However, note that the shape memory alloy causes a phase change and a change in elastic modulus at a predetermined temperature T as a boundary. The predetermined temperature T is higher than a room temperature. The SMA tube 2 causes a phase change at the predetermined temperature T, and an elastic modulus in a case where the temperature is equal to or higher than the predetermined temperature T is higher than an elastic modulus in a case where the temperature is lower than the predetermined temperature T.

In other words, rigidity of a portion of the SMA tube 2 at a temperature equal to or higher than the predetermined temperature T is higher than rigidity of a portion at a temperature lower than the predetermined temperature T. Here, the rigidity indicates the resistance to bending deformation of the longitudinal axis of the SMA tube 2. The rigidity is represented as a force needed to bend a section of the SMA tube 2 with a predetermined length in the direction along the longitudinal axis at a predetermined curvature. As the rigidity is higher, it is more difficult for the SMA tube 2 to cause the deformation in a bending direction.

In the following description, an end of both ends of the SMA tube 2 in the axial direction located on the side of the one end 102a of the insertion portion 102 will be referred to as a distal end 2a, and an end located on the side of the other end 102b of the insertion portion 102 will be referred to as a proximal end 2b. As will be described later, the SMA tube 2 serves as a conduit through which the refrigerant flows from the side of the distal end 2a toward the side of the proximal end 2b.

The distal end 2a of the SMA tube 2 in the present embodiment is closed. Note that although FIG. 3 illustrates a member that closes the distal end 2a as the same member as the SMA tube 2, the member that closes the distal end 2a may be another member that is different from the SMA tube 2.

A discharge port 2d that is connected to a return conduit 122, which will be described later, is provided at the proximal end 2b of the SMA tube 2. The inner space of the SMA tube 2 communicates with inside of the return conduit 122 via the discharge port 2d. The return conduit 122 is connected to the feeding device 120.

The heater 3 heats a heated section B that is a predetermined section of the SMA tube 2 in the axial direction. The heated section B may be a part or an entirety of the SMA tube 2. The heater 3 is a heating wire that generates heat through power distribution, for example. In an example of the present embodiment, the heater 3 is disposed along an outer periphery of the heated section B of the SMA tube 2.

Note that the heater 3 may be in direct contact with the SMA tube 2 or may sandwich a heat transmitting member with the SMA tube 2. The heater 3 may be disposed inside the SMA tube 2. The number of the heaters 3 included in the rigidity variable device 1 may be one or more.

The heater 3 can operate and thereby heat the heated section B of the SMA tube 2 to the predetermined temperature T or higher. The heater 3 is electrically connected to the control unit 130, and operations of the heater 3 are controlled by the control unit 130.

In a case in which the first switch 111 of the operation portion 110 is in an ON state, the control unit 130 causes the heater 3 to operate. In a case in which the first switch 111 of the operation portion 110 is in an OFF state, the control unit 130 stops the operation of the heater 3. As described above, electric power needed to operate the heater 3 is supplied from the external device to which the insertion device 100 is connected. As described above, the portion of the SMA tube 2 heated to the predetermined temperature T or higher through the operation of the heater 3 has higher rigidity.

The refrigerant supply tube 4 is a pipe with flexibility. The refrigerant supply tube 4 is disposed along the longitudinal axis A inside the flexible tube portion 102c of the insertion portion 102. More specifically, the refrigerant supply tube 4 in the present embodiment is inserted into the SMA tube 2. The refrigerant supply tube 4 deforms with bending deformation of the flexible tube portion 102c. In the following description, an end of both ends of the refrigerant supply tube 4 in the axial direction on the side of the one end 102a of the insertion portion 102 will be referred to as a distal end 4a, and an end on the side of the other end 102b of the insertion portion 102 will be referred to as a proximal end 4b.

The distal end 4a of the refrigerant supply tube 4 is disposed in the vicinity of the distal end 2a of the SMA tube 2. An ejection port 4c is provided at the distal end 4a of the refrigerant supply tube 4. The ejection port 4c is an opening that communicates with inside of the distal end 4a of the SMA tube 2. In other words, the refrigerant supply tube 4 and the SMA tube 2 are connected on the side of the distal ends. As will be described later, the refrigerant supply tube 4 is a conduit through which the refrigerant flows from the side of the proximal end 4b toward the side of the distal end 4a and supplies the refrigerant to the distal end 2a of the SMA tube 2.

A connection port 4d that is connected to the feeding device 120 is provided at the proximal end 4b of the refrigerant supply tube 4. The connection port 4d is connected to the feeding device 120 via a feeding conduit 121 in one example of the present embodiment.

It is possible to reduce the size of the rigidity variable device 1 in the present embodiment by disposing the refrigerant supply tube 4 inside the SMA tube 2. The rigidity variable device 1 in the present embodiment has one columnar-shaped appearance that is elongated in the axial direction and is thus easily disposed inside the insertion portion 102 of the insertion device 100.

The feeding device 120 is an electric pump that feeds the refrigerant. Although the type of the refrigerant is not particularly limited, the refrigerant in the present embodiment is a liquid such as water. The feeding device 120 operates and thereby feeds the refrigerant to the inside of the connection port 4d of the refrigerant supply tube 4.

The feeding device 120 is electrically connected to the control unit 130, and operations of the feeding device 120 are controlled by the control unit 130. In a case in which the second switch 112 of the operation portion 110 is in an ON state, the control unit 130 causes the feeding device to operate. In a case in which the second switch 112 of the operation portion 110 is in an OFF state, the control unit 130 stops the operation of the feeding device 120. As described above, the electric power needed to operate the feeding device 120 is supplied from the external device to which the insertion device 100 is connected.

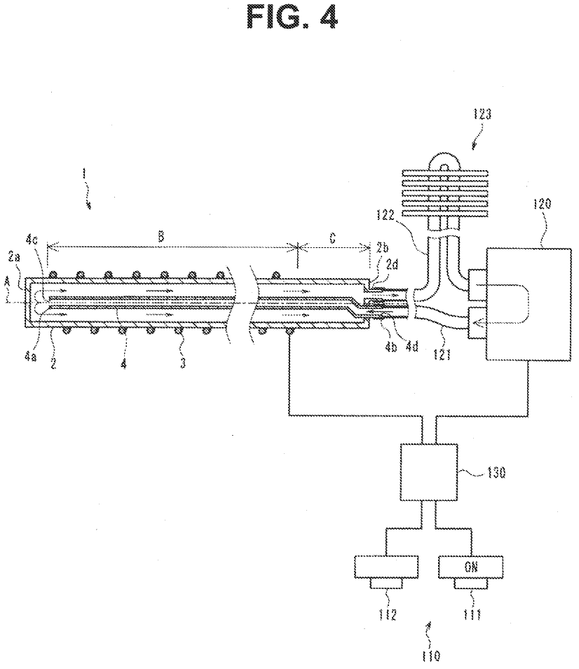

If the feeding device 120 operates, then the refrigerant is fed from the connection port 4d to the inside of the refrigerant supply tube 4 and flows inside the refrigerant supply tube 4 from the proximal end 4b toward the distal end 4a as illustrated by the arrow in FIG. 4. Then, the refrigerant is ejected from the ejection port 4c disposed inside the distal end 2a of the SMA tube 2.

Here, since the distal end 2a of the SMA tube 2 is closed, a flowing direction of the refrigerant ejected from the ejection port 4c is reversed, and the refrigerant then flows inside the SMA tube 2 from the distal end 2a toward the proximal end 2b.

Then, the refrigerant is discharged to outside of the SMA tube 2 via the discharge port 2d provided at the proximal end 2b of the SMA tube 2. The refrigerant discharged from the discharge port 2d is fed into the feeding device 120 via the return conduit 122. In an example of the present embodiment, the return conduit 122 is provided with a heat radiator 123.

Note that although the refrigerant flows to circulate through the feeding device 120, the refrigerant supply tube 4, and the SMA tube 2 through the operation of the feeding device 120 in the present embodiment, a form in which the refrigerant does not circulate may also be employed. In this case, the refrigerant is supplied from a reservoir tank or the like outside the insertion device 100 and is then discharged to the outside of the insertion device 100 via the return conduit 122.

In this manner, the rigidity variable device 1 in the present embodiment is configured to perform flowing-in and discharge of the refrigerant on the side of the proximal end 2b of the SMA tube 2 by folding back the flowing direction of the refrigerant on the side of the distal end 2a of the SMA tube 2. Therefore, there is no need to dispose configurations such as an opening portion and a conduit that handle the refrigerant in the vicinity of the one end 102a of the insertion portion 102 of the insertion device 100, and it is thus possible to reduce the size in the vicinity of the one end 102a of the insertion portion 102.

Operations of the insertion device 100 with the configuration as described above will be described.

In a case of a first state in which the first switch 111 of the operation portion 110 is in an OFF state and the second switch 112 is in an ON state or an OFF state, the heater 3 does not operate, and the temperature of the heated section B of the SMA tube 2 is lower than the predetermined temperature T. Therefore, the rigidity of the entire heated section B of the SMA tube 2 is low in the first state.

In a second state in which the first switch 111 of the operation portion 110 is in an ON state and the second switch 112 is in an OFF state, the heater 3 operates while the feeding device 120 does not operate. In the second state, the refrigerant does not flow, and the temperature of the entire heated section B of the SMA tube 2 is thus equal to or higher than the predetermined temperature T. Therefore, the rigidity of the entire heated section B of the SMA tube 2 in the second state is higher than the rigidity in the first state.

In other words, when the state is shifted from the first state to the second state in the insertion device 100 according to the present embodiment, rigidity of the portion of the flexible tube portion 102c in which the SMA tube 2 (rigidity variable device 1) is disposed increases. Here, the rigidity of the flexible tube portion 102c indicates the resistance to bending deformation of the longitudinal axis A of the flexible tube portion 102c, similarly to the aforementioned rigidity of the SMA tube 2. As the rigidity is higher, it is more difficult to cause deformation of the flexible tube portion 102c in the bending direction.

Since the refrigerant flows inside the SMA tube 2 if the second switch 112 is brought into an ON state in a case in which the second state is shifted to the first state in the insertion device 100 according to the present embodiment, it is possible to quickly lower the temperature of the heated section B to a temperature lower than the predetermined temperature T and cool the SMA tube 2. In other words, according to the insertion device 100 in the present embodiment, it is possible to quickly change the rigidity in the portion of the flexible tube portion 102c in which the SMA tube 2 is disposed into a low state at the time of the shifting from the second state to the first state.

In a case of a third state in which both the first switch 111 and the second switch 112 of the operation portion 110 are in an ON state, the heater 3 and the feeding device 120 operate.

In the third state, the refrigerant flows inside the SMA tube 2 from the distal end 2a toward the proximal end 2b in a state in which the heated section B of the SMA tube 2 is heated. In the third state, heat moves from the distal end 2a toward the proximal end 2b of the SMA tube 2 due to the flow of the refrigerant, and the temperature of the heated section B of the SMA tube 2 thus becomes higher toward the proximal end 2b.

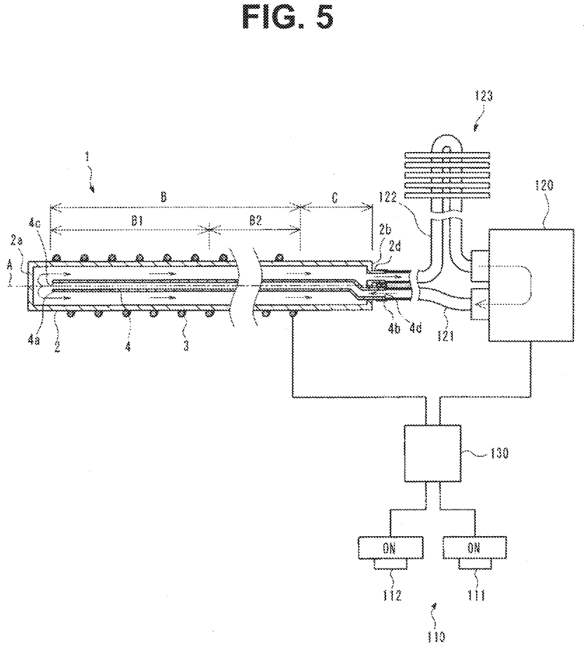

Therefore, in the third state, a temperature of a proximal end-side heated section B2, which is a partial section of the heated section B of the SMA tube 2 closer to the proximal end 2b, is equal to or higher than the predetermined temperature T while temperature of a distal end-side heated section B1, which is a remaining section closer to the distal end 2a, is lower than the predetermined temperature T as illustrated in FIG. 5. Therefore, in the third state, the rigidity of the distal end-side heated section B1 of the heated section B of the SMA tube 2 closer to the distal end 2a is in a low state similarly to the first state, and the rigidity of the proximal end-side heated section B2 closer to the proximal end 2b is higher than the rigidity of the distal end-side heated section B1.

Note that the boundary between the distal end-side heated section B1 and the proximal end-side heated section B2 in the third state can be moved in the axial direction by changing one or both of the amount of heat generated by the heater 3 and the flow amount of the refrigerant.

According to the insertion device 100 in the present embodiment, a non-heated section C (illustrated in FIG. 5) that is a section on the side of the proximal end 2b beyond the heated section B of the SMA tube 2 can be heated to a predetermined temperature T or higher using the heat of the refrigerant in the third state. The non-heated section C is a section on the side of the proximal end 2b beyond the heated section B, in a section of the SMA tube 2 to which the heater 3 is not adjacent.

In this manner, according to the insertion device 100 in the present embodiment, it is possible to increase only rigidity of a part closer to the proximal end in the portion of the flexible tube portion 102c in which the SMA tube 2 is disposed by achieving the third state.

Therefore, the insertion device 100 can cause the portion of the flexile tube portion 102c at which rigidity is to be increased to move to the side of the other end 102b in accordance with the shift from the first state to the third state. More specifically, in the first state, rigidity of the portions of the flexible tube portion 102c in which the distal end-side heated section B1 and the proximal end-side heated section B2 are disposed increases. On the other hand, in the third state, rigidity of at least one of the proximal end-side heated section B2 and the non-heated section C in the flexible tube portion 102c, increases.

In a case in which the insertion portion 102 is inserted into an intestinal tract of a human body, for example, it becomes easy to move the insertion portion 102 in the axial direction in the intestinal tract if rigidity at a portion of the intestinal tract located at a curved portion is increased. The insertion device 100 according to the present embodiment can cause the position of the insertion portion 102 at which the rigidity is to be increased to move by the shift of the first state and the third state in accordance with the position of the insertion portion 102 in the intestinal tract and can thus easily move the insertion portion 102.

### Second Embodiment

Hereinafter, a second embodiment of the invention will be described. Only different points from the first embodiment will be described below, the same reference signs will be applied to components similar to the components in the first embodiment, and description of the similar components will appropriately be omitted.

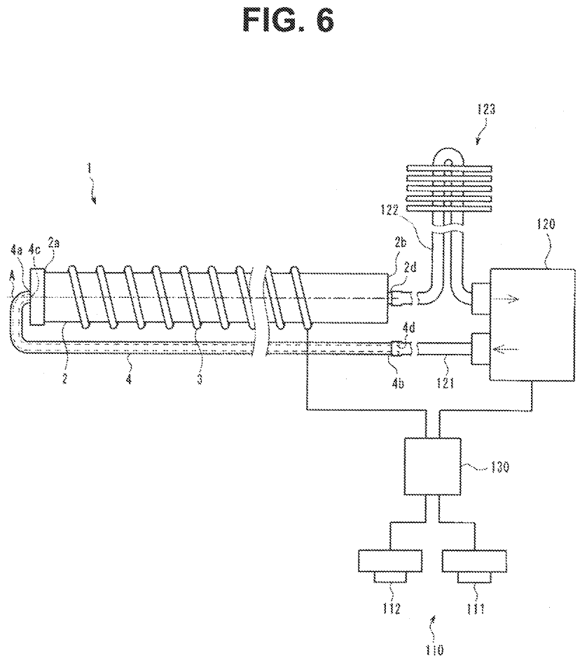

An insertion device 100 according to the present embodiment is different from the insertion device 100 in the first embodiment in a configuration of a rigidity variable device 1. FIG. 6 is a diagram illustrating a configuration of the rigidity variable device 1 according to the present embodiment.

The rigidity variable device 1 according to the embodiment is different from the rigidity variable device 1 in the first embodiment in that a refrigerant supply tube 4 is disposed outside an SMA tube 2. An ejection port 4c is provided at a distal end 4a of the refrigerant supply tube 4, and a connection port 4d is provided at a proximal end 4b, which are similar to the first embodiment.

Therefore, a refrigerant fed from a feeding device 120 flows inside the refrigerant supply tube 4 from the proximal end 4b toward the distal end 4a, is then ejected from the ejection port 4c to a distal end 2a inside the SMA tube 2, and further flows inside the SMA tube 2 from the distal end 2a toward a proximal end 2b in the present embodiment as well, similarly to the first embodiment. Therefore, the insertion device 100 according to the present embodiment can cause a portion of a flexible tube portion 102c in which rigidity is to be increased to move to the side of the other end 102b in accordance with the shift from the first state to the third state.

The invention is not limited to the aforementioned embodiments and can appropriately be modified without departing from the gist or the idea of the invention that can be read from the claims and the entire specification, and insertion devices including such modifications are also included within the technical scope of the invention.

What is claimed is:

1. An insertion device comprising:
an insertion portion including one end and another end and configured to be inserted into an insertion target from a side of the one end;
a shape memory alloy tube including a distal end and a proximal end and provided in the insertion portion such that the distal end is located on the side of the one end of the insertion portion and the proximal end is located on a side of the other end of the insertion portion, the shape memory alloy tube being configured to serve as a conduit through which a refrigerant flows from a side of the distal end toward a side of the proximal end;
a heater configured to heat the shape memory alloy tube; and
a refrigerant supply tube configured to supply the refrigerant to the distal end of the shape memory alloy tube by causing the refrigerant to flow from the side of the proximal end of the shape memory alloy tube toward the side of the distal end of the shape memory alloy tube,
wherein the refrigerant supply tube is disposed inside the shape memory alloy tube.

2. The insertion device according to claim 1, wherein the distal end of the shape memory alloy tube is closed.

3. The insertion device according to claim 1, further comprising:
a feeding device configured to feed the refrigerant to an inside of the refrigerant supply tube; and
an operation portion to which a user inputs instructions for operations of the heater and the feeding device.

4. An endoscope comprising:
an insertion portion including one end and another end and configured to be inserted into an insertion target from a side of the one end;
a shape memory alloy tube including a distal end and a proximal end and provided in the insertion portion such that the distal end is located on the side of the one end of the insertion portion and the proximal end is located on a side of the other end of the insertion portion, the shape memory alloy tube being configured to serve as a conduit through which a refrigerant flows from a side of the distal end toward a side of the proximal end;
a heater configured to heat the shape memory alloy tube; and
a refrigerant supply tube configured to supply the refrigerant to the distal end of the shape memory alloy tube by causing the refrigerant to flow from the side of the proximal end of the shape memory alloy tube toward the side of the distal end of the shape memory alloy tube,
wherein the refrigerant supply tube is disposed inside the shape memory alloy tube.
